SEQUENCE LISTING (23 sequences)

SEQ ID NO: 1 - DNA, 201 bp, Homo sapiens
  variation (101): n=A or G, polymorphic site
tctataccacagtagtgttgatctcaagagcttagcatgttggcttaaagacatccagggatgaggtgttggagtcagattgagtttgaatcttagctctncttgtattactgtgttatcttggccaagtatttaacctctctgaaataggttttctcagggctgtgaagtttggaagatacataaaagcccaaagaaaaa

SEQ ID NO: 2 - DNA, 201 bp, Homo sapiens
  variation (101): n=C or G, polymorphic site
tgacctcaggtgatccacccacctcggcatcccaaagtgctgggattacaggtgtgagccaccacaccaggccttgggtaccatgccttgaaccatttcantgccttttggagatggatgagttgccagcatccttcttagatccctatatatttgtttatttattgaacaaatacatattaacttatctttttgaccaaa

SEQ ID NO: 3 - DNA, 201 bp, Homo sapiens
  variation (101): n=A or C, polymorphic site
tgctcatgtaccttatggatttgacccacctcattctggacaaancctcaggaggatctcttcagggacatgatgcagttttgagactggtggagattcgnacggtatgaagcatttggcttcttggagttttaggtttctaaattttgagctccaagggtatcacacagtagctctcatttaagtgagtcttctcatgtt

SEQ ID NO: 4 - DNA, 201 bp, Homo sapiens
  variation (101): n=A or G, polymorphic site
aattggtaattactttgattaaattaatttaaaacttggcagtctgtggagacatttttgattgttagagcttggaggggccatccgtgggaagaggccanggatgctgctggaaacctacaatgcccaggacagccctcaacaaaaaatgttctggcttcaaatgtcaatagctctgagattgagaaaccctgttatagt

SEQ ID NO: 5 - DNA, 201 bp, Homo sapiens
  variation (101): n=A or G, polymorphic site
aagcagaagatgaccagtctgaggcttcagggaagaaatctgtgaagggagtgtctaagaaatatgttcctccacgcttggttccagtacattatggtatnaactttggctgctgcctcctcagcatgaactgtttctcttttctctgttcttggataaccctgcttattttcatcatgtagatgaaacagaagctgagcg

SEQ ID NO: 6 - DNA, 21 bp, Artificial Sequence
  primer
atggcggcgctgggggtgctg

SEQ ID NO: 7 - DNA, 21 bp, Artificial Sequence
  primer
tcaccgccgcctccgaaaacc

SEQ ID NO: 8 - DNA, 30 bp, Artificial Sequence
  primer
acgttggatgaaagacatccagggatgagg

SEQ ID NO: 9 - DNA, 30 bp, Artificial Sequence
  primer
acgttggatgacttggccaagataacacag

SEQ ID NO: 10 - DNA, 20 bp, Artificial Sequence
  primer
tgagtttgaatcttagctct

SEQ ID NO: 11 - DNA, 30 bp, Artificial Sequence
  primer
acgttggatggggatctaagaaggatgctg

SEQ ID NO: 12 - DNA, 30 bp, Artificial Sequence
  primer
acgttggatgttgggtaccatgccttgaac

SEQ ID NO: 13 - DNA, 17 bp, Artificial Sequence
  primer
tccatctccaaaaggca

SEQ ID NO: 14 - DNA, 30 bp, Artificial Sequence
  primer
acgttggatgactgtgtgatacccttggag

SEQ ID NO: 15 - DNA, 30 bp, Artificial Sequence
  primer
acgttggatgtgcagttttgagactggtgg

SEQ ID NO: 16 - DNA, 19 bp, Artificial Sequence
  primer
gccaaatgcttcataccgt

SEQ ID NO: 17 - DNA, 30 bp, Artificial Sequence
  primer
acgttggatgtcctgggcattgtaggtttc

SEQ ID NO: 18 - DNA, 30 bp, Artificial Sequence
  primer
acgttggatggattgttagagcttggaggg

SEQ ID NO: 19 - DNA, 20 bp, Artificial Sequence
  primer
gtaggtttccagcagcatcc

SEQ ID NO: 20 - DNA, 30 bp, Artificial Sequence
  primer
acgttggatgagaaatatgttcctccacgc

SEQ ID NO: 21 - DNA, 30 bp, Artificial Sequence
  primer
acgttggatgaacagttcatgctgaggagg

SEQ ID NO: 22 - DNA, 21 bp, Artificial Sequence
  primer
ggttccagtacattatggtat

SEQ ID NO: 23 - protein, 315 aa, Homo sapiens
MAALGVLESDLPSAVTLLKNLQEQVMAVTAQVKSLTQKVQAGAYPTEKGLSFLEVKDQLLLMYLMDLTHLILDKASGGSLQGHDAVLRLVEIRTVLEKLRPLDQKLKYQIDKLIKTAVTGSLSENDPLRFKPHPSNMMSKLSSEDEEEDEAEDDQSEASGKKSVKGVSKKYVPPRLVPVHYDETEAEREKKRLERAKRRALSSSVIRELKEQYSDAPEEIRDARHPHVTRQSQEDQHRINYEESMMVRLSVSKREKGRRKRANVMSSQLHSLTHFSDISALTGGTVHLDEDQNPIKKRKKIPQKGRKKKGFRRRR

(19) United States
(12) Patent Application Publication
(10) Pub. No.: US 2009/0258345 A1
(43) Pub. Date: Oct. 15, 2009

(54) PROTEIN ASSOCIATED WITH COLORECTAL CANCER, POLYNUCLEOTIDE INCLUDING SINGLE-NUCLEOTIDE POLYMORPHISM ASSOCIATED WITH COLORECTAL CANCER, MICROARRAY AND DIAGNOSTIC KIT INCLUDING THE SAME, AND METHOD OF DIAGNOSING COLORECTAL CANCER USING THE SAME

(75) Inventors: Yeon-Su Lee, Gyeonggi-do (KR); Sang-Hoon Kim, Seoul (KR); Choon-Ryoul Shin, Seoul (KR); Kyung-Hee Park, Seoul (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si, Gyeonggi-do (KR)

(21) Appl. No.: 11/576,650

(22) PCT Filed: Feb. 25, 2006
(86) PCT No.: PCT/KR06/00665

§ 371 Date: Apr. 4, 2007

(30) Foreign Application Priority Data
Feb. 26, 2005 (KR) 10-2005-0016259

(51) Int. Cl. C12Q 1/68; C07K 14/00; G01N 33/68; C12Q 1/02; C12N 15/11; C40B 40/08

(52) U.S. Cl. 435/6; 530/350; 436/86; 435/29; 536/23.5; 536/24.33; 536/24.31; 506/17

(57) ABSTRACT

Provided are an isolated nucleolar protein having an amino acid sequence of NCBI GenBank Accession No. XP_033371, a method of diagnosing colorectal cancer in an individual, including measuring an expression level of a protein having an amino acid sequence of NCBI GenBank Accession No. XP_033371 in the individual, and a polynucleotide for diagnosis or treatment of colorectal cancer including at least 10 contiguous nucleotides of a nucleotide sequence selected from the group consisting of nucleotide sequences of SEQ ID NOS: 1-5 and including a nucleotide at position 101 of the nucleotide sequence, or a complementary polynucleotide thereof.

### TECHNICAL FIELD

The present invention relates to a protein and a polynucleotide associated with colorectal cancer, a microarray and a diagnostic kit including the same, and a method of diagnosing colorectal cancer.

### BACKGROUND ART

### 1. Incidence of Colorectal Cancer

Incidence of colorectal cancer has increased in American and European persons who frequently consume meat or other foods containing animal fat. In particular, in America, colorectal cancer is the second common cancer in both incidence and death rate. Colorectal cancer incidence in Asian countries including Korea and Japan is lower than that in Western countries but has recently increased due to rapid Westernization of diet. According to a recent report (1997), in Korea, colorectal cancer is the fourth common cancer following stomach cancer and breast cancer. Like other cancers affecting other organs, colorectal cancer frequently occurs in adults over 50 years of age but can strike younger people.

### 2. Causative and Risk Factors of Colorectal Cancer

The exact cause of colorectal cancer is not known. However, it is well known that familial adenomatous polyposis, idiopathic nonspecific ulcerative colitis, colonic polyp, and rectal polyp, in particular, villous adenoma can turn to cancer. Although there is no conclusive evidence of a hereditary link to colorectal cancer, it is suspected that about 10-30% of colorectal cancer cases are dominated by a hereditary factor.

The incidence of colorectal cancer is more frequent in Western people than in Eastern people. Such an increased incidence of colorectal cancer is suspected to be associated with higher consumption of animal fat and meat in Western diets. That is, consumption of animal fat and meat produces less stool and the stool also stays in the large intestine for a longer time, relative to consumption of fiber-rich foods such as vegetables or grains. Higher consumption of animal fat affects bacteria that normally live in the healthy large intestine. Furthermore, if the stool stays in the large intestine for a long time, carcinogens are easily generated in the large intestine and thus greater exposure of colorectal cells to the carcinogens is caused. This explains the increased incidence of colorectal cancer. Epidemiological studies reveal that there is a relationship between the consumption of animal fat and meat and the incidence of colorectal cancer.

### 3. Symptoms of Colorectal Cancer

Colorectal cancer has no specific symptoms. However, colorectal cancer involves various symptoms according to the affected region or the level of advancement, in addition to common cancer symptoms such as weight loss. For example, when cancer is caused in the descending colon adjacent to the anus, the sigmoid colon, or the rectum, common symptoms include the following: blood in the stool, a change in bowel habits (repetition of diarrhea and constipation), stool narrower than usual, feeling that the bowel does not empty completely, or stomachache. When cancer is caused in the ascending colon, anemia (dizziness, vomiting, anorexia, fatigue, difficulty in breathing, etc.) due to unperceivable, chronic blood loss in the stool is caused.

In addition, as colorectal cancer develops, a gradual narrowing of the large intestine's inner passageway causes intestinal obstruction. Occasionally, abdominal tumor mass may be found, or the spread to distant organs, such as the liver or lung, may occur.

### 4. Diagnosis of Colorectal Cancer

(1) Fecal occult blood test: the fecal occult blood test is a simple screening test to detect colorectal cancer. However, since this test can have a false-positive result due to other factors, it is not an absolute test for colorectal cancer.

(2) Tumor marker assay: the tumor marker assay is a blood test that looks for a CEA (carcinoembryonic antigen). About 50% of colorectal cancer patients undergo an increase in the CEA level. However, the increase in the CEA level does not necessarily prove the existence of colorectal cancer. Nevertheless, since a high CEA level indicates a high likelihood of colorectal cancer, a precise examination is additionally required for persons with a high CEA level. CEA is also helpful in evaluating the recurrence of colorectal cancer after treatment.

(3) Barium enema examination: the barium enema examination is radiation screening and detection of colorectal cancer based on a change in the outline of the mucosal membrane of the large intestine. Since this test shows the entire outline of the large intestine, it is helpful in detecting the location of cancer before surgery.

(4) Endoscopic examination: the endoscopic examination is divided into two groups: a short endoscopic examination to view the sigmoid colon and a long endoscopic examination to view the entire large bowel including the appendix. The endoscopic examination has a higher diagnostic accuracy than the barium enema examination. The endoscopic examination is an essential test for diagnosis of colorectal cancer since it enables histological examination, and thus the final diagnosis can be made by the histological examination, and polyps can be removed.

(5) Ultrasonic and computed tomography (CT) scan of the abdomen: when colorectal cancer is diagnosed by barium enema examination or endoscopic examination, the ultrasonic and CT scan show the localized stage and distant metastasis of the colorectal cancer.

(6) CEA and Serologic Tumor Marker Assay

For early diagnosis of colorectal cancer, various proteins, including glycoproteins, had been widely studied as promising tumor marker candidates. However, colorectal cancer-specific tumor markers have not been found to date. Currently, CEA is widely used in determining an advanced stage of colorectal cancer before surgery and evaluating the recurrence of colorectal cancer after surgery. However, CEA is not suitable for cancer patients with no symptoms.

### 5. Stage and Treatment of Colorectal Cancer

According to the Dukes' classification, the stage of colorectal cancer is classified as A, B, C, and D according to the degree of invasion into the mucosal membrane of the large intestine, the degree of lymph node metastasis, and whether it has spread to other distant organs. Like other cancers, the stage of colorectal cancer is determined after surgery, and the treatment and prognosis of colorectal cancer vary according to the stage of colorectal cancer.

(1) Endoscopic Treatment

Currently, endoscopic examination is regarded as an essential test for diagnosis of colorectal cancer, and at the same time, plays an important role in prevention or treatment of colorectal cancer. During endoscopic examination, polyps that may develop into cancer can be removed, thereby reducing the incidence of colorectal cancer. At the same time, colorectal cancer patients with small tumor mass like polyps can be simply treated by endoscopic resection.

(2) Surgical Treatment

Surgery is a primary treatment for colorectal cancer and has a significant effect on the treatment result. The surgical treatment depends on the region affected by cancer. For colon cancer, the affected colon and surrounding lymph nodes are removed, and the remaining sections of the colon are then re-connected. For rectal cancer, if rectal cancer is located far away from the anus, only the cancer is removed with no removal of the anus. On the other hand, if rectal cancer is located close to the anus, the anus is removed with the cancer and an artificial anus is reconstructed.

(3) Radiotherapy

For rectal cancer, radiotherapy, together with drug therapy, may be performed after surgery according to the stage of the cancer. The radiotherapy may be given five days a week for 5-6 weeks and can reduce the risk of local recurrence and lymph node metastasis in the pelvis.

(4) Drug Therapy

After surgery, when colorectal cancer is diagnosed to be in stage B, drug therapy is used in some eases. However, since the drug therapy for the stage B colorectal cancer is not a standard treatment, surgery may be followed by only periodic observation and examination. However, for stage C colorectal cancer, drug therapy for six months to one year is used as standard treatment. For colorectal cancer at a stage D (terminal stage), drug therapy is used in spite of remarkably insignificant therapeutic effect since other therapies have failed.

### 6. Treatment Result

The 5-year survival rate for colorectal cancer after surgery is as follows: 90% for stage A, 80% for stage B, 45% for stage C, and less than 10% for stage D. Like other cancers, the 5-year survival rate for colorectal cancer is greatly reduced as colorectal cancer advances. Therefore, early diagnosis and treatment of colorectal cancer are very important.

### 7. Prevention

An exact cause of colorectal cancer (colon cancer and rectal cancer) has not been found. It is known that high consumption of animal fat or meat is probably associated with an increased risk of colorectal cancer. Thus, a reduced intake of animal fat and a balanced diet of fresh vegetables and fiber-rich foods are recommended. Furthermore, it is necessary to avoid high consumption of foods containing chemicals such as dark pigments and preservatives.

When diseases closely associated with colorectal cancer, i.e., familial adenomatous polyposis, idiopathic nonspecific ulcerative colitis, colonic polyp, and rectal polyp are found, much interest and periodic examination are required to prevent colorectal cancer.

As described above, CEA is generally known as a colorectal cancer-specific marker. However, CEA has many limitations in early diagnosis of colorectal cancer.

C14orf120 (NCBI GenBank Accession No.: XP_{—}033371) is human chromosome 14 open reading frame 120 and its function is not known. According to a computer-mediated automatic analysis result, the protein C14orf120 contains Sas10 and Utp3 belonging to Sas10/Utp3 family. However, the accurate functions of this family are not known. It is known that gene c14orf120 is present in band 14q11.2.

About thirty single-nucleotide polymorphisms (SNPs) were observed in the human c14orf120 gene. However, no relationship between these SNPs and rectal cancer has been found. SNP is a form of genetic variations in living species. Different types of polymorphisms are known, including restriction fragment length polymorphisms (RFLPs), short tandem repeats (STRs), variable number tandem repeats (VNTRs) and single-nucleotide polymorphisms (SNPs). Among them, SNPs take the form of single-nucleotide variations between individuals of the same species. When SNPs occur in protein coding sequences, any one of the polymorphic forms may give rise to the expression of a defective or a variant protein. On the other hand, when SNPs occur in non-coding sequences, some of these polymorphisms may result in the expression of defective or variant proteins (e.g., as a result of defective splicing). Other SNPs have no phenotypic effects.

It is known that human SNPs appear at a frequency of 1 in about 30 bp. to 1,000 bp. When such SNPs induce the phenotypic expression such as a disease, polynucleotides containing the SNPs can be used as primers or probes for diagnosis of the disease. Currently, research into the nucleotide sequences and functions of SNPs is under way by many research institutes. The nucleotide sequences and other experimental results of the identified human SNPs have been collated into a database to be easily accessible. Even though findings available to date show that specific SNPs exist on human genomes or cDNAs, phenotypic effects of such SNPs have not been revealed. Functions of most SNPs have not yet been discovered.

As described above, no colorectal cancer-specific markers except CEA are known. In particular, it has heretofore been unknown that the protein C14orf120 can be expressed specifically in relation to colorectal cancer. Also, it has heretofore been unknown that any of genetic polymorphism on the gene c14orf120 is specifically associated with colorectal cancer.

### DISCLOSURE OF INVENTION

### Technical Problem

Therefore, while making efforts to find the function of the protein C14orf120 in cells, the present inventors found that the protein C14orf120 was associated with colorectal cancer, and several SNPs on the gene c14orf120 were associated with colorectal cancer, and thus completed the present invention.

The present invention provides an isolated protein associated with colorectal cancer.

The present invention also provides a method of diagnosing colorectal cancer using the protein.

The present invention also provides a polynucleotide containing single-nucleotide polymorphism (SNP) associated with colorectal cancer.

The present invention also provides a microarray and a diagnostic kit for the detection of colorectal cancer, each of which includes the polynucleotide containing SNP associated with colorectal cancer.

The present invention also provides a method of analyzing polynucleotides associated with colorectal cancer.

The present invention provides an isolated nucleolar protein having an amino acid sequence of NCBI GenBank Accession No.: XP_{—}033371.

The present invention also provides a method of diagnosing colorectal cancer, which includes measuring an expression amount of a nucleolar protein having an amino acid sequence of NCBI GenBank Accession No.: XP_{—}033371.

### Technical Solution

In the method of the present invention, the expression amount of the nucleolar protein may be determined by measuring the amount of nucleolar protein in cells derived from an individual or the amount of mRNA encoding the nucleolar protein. When the expression amount of the nucleolar protein is 20% or more higher than that in normal cells, it may be determined that the individual has a higher likelihood of being diagnosed as a colorectal cancer patient or as at risk of developing colorectal cancer. However, the present invention is not limited thereto.

The nucleolar protein of NCBI GenBank Accession No.: XP_{—}033371 is conventionally known as C14orf120 which is human chromosome 14 open reading frame 120 and its function is not known. According to a computer-mediated automatic analysis result, the protein of NCBI GenBank Accession No.: XP_{—}033371 contains Sas10 and Utp3 belonging to Sas10/Utp3 family. However, the accurate functions of this family are not known. The amino acid sequence of XP_{—}033371 is as set forth in SEQ ID NO: 13.

The present inventors measured an expression level of the protein of NCBI GenBank Accession No.: XP_{—}033371 both in normal cells and in tumor cells, and found that the protein of NCBI GenBank Accession No.: XP_{—}033371 exhibited a greatly increased expression level, in particular, in colorectal cancer cells, relative to in normal cells and other cancer cells. FIGS. 1 and 2 show that the protein of NCBI GenBank Accession No: XP_{—}033371 of the present invention is expressed at a remarkably high level in colorectal cancer cells, relative to other cancer cells and normal cells.

The present inventors also isolated and cloned a gene of the protein of NCBI GenBank Accession No: XP_{—}033371 from SNU-449 cell lines, cloned a fusion gene of it with a gene encoding a GFP protein, and transfected the cloned products into osteosarcoma cell lines (U2OS), to identify an expression position in cells. As a result, it was identified that the protein of NCBI GenBank Accession No: XP_{—}033371 of the present invention was present in nucleoli during interphase and mitosis. FIGS. 3 and 4 show that the protein of NCBI GenBank Accession No: XP_{—}033371 is expressed in nucleoli during interphase and mitosis. FIG. 5 shows the expression of the protein of NCBI GenBank Accession No: XP_{—}033371 detected in nucleoli using an antibody against nucleolar protein B23. It is found that the protein of NCBI GenBank Accession No: XP_{—}033371 of the present invention is associated with disassembly of nucleoli. FIG. 6 shows that a GFP-XP_{—}033371 fusion protein is associated with disassembly of nucleoli.

In addition, a protein interacting with the protein of NCBI GenBank Accession No: XP_{—}033371 was investigated using a yeast two-hybrid system. As a result, it is found that the protein of NCBI GenBank Accession No: XP_{—}033371 interacts with proteins presented in Table 1 below.

TABLE 1

| Sequence | | Sequenced | | | Coding |
|---|---|---|---|---|---|
| name | Gene | region | Remark | State | sequence |
| C2 | | | | Failure | |
| C3 | Myc-binding | 179-2332 | hypothetical | Good | 75-2918 |
| | protein- | | protein | | |
| | associated | | | | |
| | protein | | | | |
| C4 | YB-1 | 839-1500 | | Good | 115-1089 |
| C5 | AATF | 271-1051 | Apoptosis | Good | 180-1862 |
| | | | antagonizing | | |
| | | | transcription | | |
| | | | factor | | |
| C6 | AATF | 223-1011 | Apoptosis | Good | 180-1862 |
| | | | antagonizing | | |
| | | | transcription | | |
| | | | factor | | |
| C8 | Myc-binding | 2493-2988 | AMY1- | Good | 75-2918 |
| | protein- | | associated | | |
| | associated | | protein 1; | | |
| | protein | | Myc-binding | | |
| | | | protein- | | |
| | | | associated | | |
| | | | protein | | |
| C10 | Myc-binding | 2746-2988 | AMY1- | Good | 75-2918 |
| | protein- | | associated | | |
| | associated | | protein 1; | | |
| | protein | | Myc-binding | | |
| | | | protein- | | |
| | | | associated | | |
| | | | protein | | |
| C11 | AATF | 271-1054 | Apoptosis | Good | 180-1862 |
| | | | antagonizing | | |
| | | | transcription | | |
| | | | factor | | |
| C12 | AATF | 214-994 | Apoptosis | Good | 180-1862 |
| | | | antagonizing | | |
| | | | transcription | | |
| | | | factor | | |
| C14 | AATF | 271-1046 | Apoptosis | Good | 180-1862 |
| | | | antagonizing | | |
| | | | transcription | | |
| | | | factor | | |
| C15 | Myc-binding | 2743-2988 | AMY1- | Good | 75-2918 |
| | protein- | | associated | | |
| | associated | | protein 1; | | |
| | protein | | Myc-binding | | |
| | | | protein- | | |
| | | | associated | | |
| | | | protein | | |
| C16 | AATF | 17-774 | Apoptosis | | 180-1862 |
| | | | antagonizing | | |
| | | | transcription | | |
| | | | factor | | |
| C17 | AATF | 262-1043 | Apoptosis | Good | 180-1862 |
| | | | antagonizing | | |
| | | | transcription | | |
| | | | factor | | |
| C18 | Myc-binding | 2746-2988 | AMY1- | Good | 75-2918 |
| | protein- | | associated | | |
| | associated | | protein 1; | | |
| | protein | | Myc-binding | | |
| | | | protein- | | |
| | | | associated | | |
| | | | protein | | |
| C19 | Myc-binding | 2743-2988 | AMY1- | Good | 75-2918 |
| | protein- | | associated | | |
| | associated | | protein 1; | | |
| | protein | | Myc-binding | | |
| | | | protein- | | |
| | | | associated | | |
| | | | protein | | |
| C20, C21 | Myc-binding | 2538-2988 | AMY1-assoc | Good | 75-2918 |
| | protein- | | associated | | |
| | associated | | protein 1; | | |
| | protein | | Myc-binding | | |
| | | | protein- | | |
| | | | associated | | |
| | | | protein | | |
| C23 | AATF | 223-1051 | Apoptosis | Good | 180-1862 |
| | | | antagonizing | | |
| | | | transcription | | |
| | | | factor | | |
| C29 | | | | Failure | |
| C30 | AATF | 587-1415 | Apoptosis | Good | 180-1852 |
| | | | antagonizing | | |
| | | | transcription | | |
| | | | factor | | |
| C34 | | | | Failure | |
| C36 | | | | Failure | |
| C37 | AATF | 17-835 | Apoptosis | Good | 180-1862 |
| | | | antagonizing | | |
| | | | transcription | | |
| | | | factor | | |
| C38 | C1QBP | 216-1032 | Complement | Good | 22-870 |
| | | | component 1, | | |
| | | | q sub- | | |
| | | | component | | |
| | | | binding | | |
| | | | protein | | |

As shown in Table 1, total 18 positive colonies, i.e, C1QBP1, YB-1, ten AATFs, and six Myc-binding protein-associated proteins were found.

The above results reveal that the protein of NCBI GenBank Accession No: XP_{—}033371 is present in nucleoli and has a nucleolus-associated function. Judging from the fact that the protein is present in chromosome during mitosis, the protein has a function related to cell cycle. In addition, AATF and the protein of NCBI GenBank Accession No: XP_{—}033371 are functionally associated with each other. It is known that AATF is a tumor protein binding with RB and inhibiting the growth inhibitory effect of RB. Thus, the protein of NCBI GenBank Accession No: XP_{—}033371 of the present invention binds with AATF to facilitate the binding of AATF with RB or cooperates with AATF to thereby induce tumorigenesis.

The present invention provides a polynucleotide for diagnosis or treatment of colorectal cancer including at least 10 contiguous nucleotides of a nucleotide sequence selected from the group consisting of nucleotide sequences of SEQ ID NOS: 1-5 derived from c14orf120 gene and including a nucleotide of a polymorphic site (position 101) of the nucleotide sequence, or a complementary polynucleotide thereof.

The polynucleotide includes at least 10 contiguous nucleotides containing a polymorphic site of a nucleotide sequence selected from the nucleotide sequences of SEQ ID NOS: 1-5. The polynucleotide is 10 to 400 nucleotides in length, preferably 10 to 100 nucleotides in length, and more preferably 10 to 50 nucleotides in length. The polymorphic site of each nucleotide sequence of SEQ ID NOS: 1-5 is at position 101.

Each nucleotide sequence of SEQ ID NOS: 1-5 is a polymorphic sequence. The polymorphic sequence refers to a nucleotide sequence containing a polymorphic site at which single-nucleotide polymorphism (SNP) occurs. The polymorphic site refers to a position of the polymorphic sequence at which SNP occurs. Each nucleotide sequence of SEQ ID NOS: 1-5 may be DNA or RNA.

In the present invention, each polymorphic site (position 101) of the polymorphic sequences of SEQ ID NOS: 1-5 is associated with colorectal cancer. This is confirmed by DNA nucleotide sequence analysis of blood samples from colorectal cancer patients and normal persons. The association of the polymorphic sequences of SEQ ID NOS: 1-5 with colorectal cancer and the characteristics of the polymorphic sequences are summarized in Tables 2 and 3.

TABLE 2

| | SNP | | | | | | | | | | |
|---|---|---|---|---|---|---|---|---|---|---|---|
| Marker | sequence | | Allele frequency | | | Genotype frequency | | | | | |
| name | (SEQ ID NO.) | SNP | cas_A2 | con_A2 | Delta | cas_A1A1 | cas_A1A2 | cas_A2A2 | con_A1A1 | con_A1A2 | con_A2A2 |
| CCK061 | 1 | [A/G] | 0.646 | 0.714 | 0.068 | 31 | 101 | 98 | 22 | 120 | 145 |
| CCK162 | 2 | [G/C] | 0.647 | 0.714 | 0.067 | 31 | 99 | 98 | 21 | 120 | 142 |
| CCY_067 | 3 | [A/C] | 0.377 | 0.286 | 0.091 | 97 | 85 | 42 | 147 | 123 | 22 |
| CCY_202 | 4 | [G/A] | 0.355 | 0.285 | 0.07 | 103 | 106 | 33 | 148 | 123 | 22 |
| CCY_205 | 5 | [A/G] | 0.631 | 0.704 | 0.073 | 33 | 108 | 95 | 21 | 123 | 135 |

| | | | Odds ratio (OR): multiple model | | | | | | |
|---|---|---|---|---|---|---|---|---|---|
| Marker | df = 2 | | Risk | | | HWE status | | Call rate | |
| name | Chi_value | Chi_exact_p-Value | allele | OR | CI | con_HW | cas_HW | cas_call_rate | con_call_rate |
| CCK061 | 6.041 | 4.88E−02 | A1 A | 1.37 | (1.055, 1.785) | .569, HWE | .127, HWE | 1 | 0.98 |
| CCK162 | 6.155 | 4.61E−02 | A1 G | 1.36 | (1.044, 1.774) | .657, HWE | .419, HWE | 0.99 | 0.97 |
| CCY_067 | 14.733 | 6.32E−04 | A2 C | 1.52 | (1.164, 1.965) | 9.12, HWD | .185, HWE | 0.88 | 0.99 |
| CCY_202 | 6.729 | 3.46E−02 | A2 A | 1.39 | (1.068, 1.792) | .535, HWE | .185, HWE | 0.95 | 0.99 |
| CCY_205 | 7.056 | 2.94E−02 | A1 A | 1.39 | (1.071, 1.805) | .083, HWE | .863, HWE | 0.92 | 0.94 |

TABLE 3
characteristics of the polymorphic sequences of SEQ ID NOS: 1-5

| | | | | | | | | | Amino |
|---|---|---|---|---|---|---|---|---|---|
| Marker | | | Chromosome | Chromosome | | | | SNP | acid |
| name | rs | SNP | number | position | Band | Gene | Description | function | change |
| CCK061 | rs7151139 | [A/G] | 14 | 21933597 | 14q11.2 | C14orf120 | Chromosome | Intron | No |
| | | | | | | | 14orf120 | | change |
| CCK162 | rs10142383 | [G/C] | 14 | 21932663 | 14q11.2 | C14orf120 | Chromosome | Intron | No |
| | | | | | | | 14orf120 | | change |
| CCY_067 | rs2236261 | [A/C] | 14 | 21934642 | 14q11.2 | C14orf120 | Chromosome | Coding- | No |
| | | | | | | | 14orf120 | synon, | change |
| | | | | | | | | reference | |
| CCY_202 | rs6573195 | [G/A] | 14 | 21934148 | 14q11.2 | C14orf120 | Chromosome | Intron | No |
| | | | | | | | 14orf120 | | change |
| CCY_205 | rs2295706 | [A/G] | 14 | 21935494 | 14q11.2 | C14orf120 | Chromosome | Intron | No |
| | | | | | | | 14orf 20 | | change |

In Tables 2 and 3, the contents in columns are as defined below.
A1 and A2 represent a low mass allele and a high mass allele, respectively, as a result of sequence analysis according a homogeneous MassEXTEND (hME) technique (Sequenom), and are optionally designated for convenience of experiments.
rs represents SNP identification number assigned by NCBI GenBank.
SNP sequence represents a sequence containing a SNP site, i.e., a sequence containing allele A1 or A2 at position 101.
cas_A2, con_A2, and Delta respectively represent allele A2 frequency of a case group, allele A2 frequency of a normal group, and the absolute value of the difference between cas_A2 and con_A2. Here, cas_A2 is (genotype A2A2 frequency×2+genotype A1A2 frequency)/(the number of samples×2) in the case group and con_A2 is (genotype A2A2 frequency×2+genotype A1A2 frequency)/(the number of samples×2) in the normal group.
Genotype frequency represents the frequency of each genotype. Here, cas_A1A1, cas_A1A2, and cas_A2A2 are the number of persons with genotypes A1A1, A1A2, and A2A2, respectively, in the case group, and con_A1A1, con_A1A2, and con_A2A2 are the number of persons with genotypes A1A1, A1 A2, and A2A2, respectively, in the normal group.
df=2 represents a chi-squared value with two degree of freedom. Chi-value represents a chi-squared value and p-value is determined based on the chi-value. Chi_exact_p-value represents p-value of Fisher's exact test of chi-square test. When the number of genotypes is less than 5, results of the chi-square test may be inaccurate. In this respect, determination of more accurate statistical significance (p-value) by the Fisher's exact test is required. The chi_exact_p-value is a variable used in the Fisher's exact test. In the present invention, when the p-value ≦0.05, it is considered that the genotype of the case group is different from that of the normal group, i.e., there is a significant difference between the case group and the normal group.
With respect to risk allele, when a reference allele is A2 and the allele A2 frequency of the case group is larger than the allele A2 frequency of the normal group (i.e., cas_A2>con_A2), the allele A2 is regarded as risk allele. In an opposite case, allele A1 is regarded as risk allele.
Power 4 represents the degree of data confidence.
Odds ratio (OR) represents the ratio of the probability of risk allele in the case group to the probability of risk allele in the normal group. In the present invention, the Mantel-Haenszel odds ratio method was used. CI represents 95% confidence interval for the odds ratio and is represented by (lower limit of the confidence interval, upper limit of the confidence interval). When 1 falls under the confidence interval, it is considered that there is insignificant association of risk allele with disease.
HWE represents that the result satisfied Hardy-Weinberg Equilibrium. Here, con_HWE and cas_HWE represent degree of deviation from the Hardy-Weinberg Equilibrium in the normal group and the case group, respectively. Based on chi_value=6.63 (p-value=0.01, df=1) in a chi-square (df=1) test, a value larger than 6.63 was regarded as Hardy-Weinberg Disequilibrium (HWD) and a value smaller than 6.63 was regarded as Hardy-Weinberg Equilibrium (HWE).
Call rate represents the number of genotype-interpretable samples to the total number of samples used in experiments. Here, cas_call_rate and con_call_rate represent the ratio of the number of genotype-interpretable samples to the total number (300 persons) of samples used in the case group and the normal group, respectively. As shown in Tables 2 and 3, according to the chi-square test of the polymorphic markers of SEQ ID NOS: 1-5 of the present invention, chi_exact_p-value ranges from 6.32×10⁻⁴ to 4.88×10⁻² in 95% confidence interval. This shows that there are significant differences between expected values and measured values in allele occurrence frequencies in the polymorphic markers of SEQ ID NOS: 1-5. Odds ratio ranges from 1.36 to 1.52, which shows that the polymorphic markers of SEQ ID NOS: 1-5 are associated with colorectal cancer.

The present invention also provides an allele-specific polynucleotide for diagnosis of colorectal cancer, which is hybridized with a polynucleotide including at least 10 contiguous nucleotides containing a polymorphic site of a nucleotide sequence selected from the group consisting of nucleotide sequences of SEQ ID NOS: 1-5, or a complement thereof.

The allele-specific polynucleotide refers to a polynucleotide specifically hybridized with each allele. That is, the allele-specific polynucleotide has the ability that distinguishes nucleotides of polymorphic sites within the polymorphic sequences of SEQ ID NOS: 1-5 and specifically hybridizes with each of the nucleotides. The hybridization is performed under stringent conditions, for example, under conditions of 1M or less in salt concentration and 25° C. or more in temperature. For example, conditions of 5×SSPE (750 mM NaCl, 50 mM Na Phosphate, 5 mM EDTA, pH 7.4) and 25-30° C. are suitable for allele-specific probe hybridization.

In the present invention, the allele-specific polynucleotide may be a primer. As used herein, the term ‘primer’ refers to a single-stranded oligonucleotide that acts as a starting point of template-directed DNA synthesis under appropriate conditions, for example in a buffer containing four different nucleoside triphosphates and polymerase such as DNA or RNA polymerase or reverse transcriptase and an appropriate temperature. The appropriate length of the primer may vary according to the purpose of use, generally 15 to 30 nucleotides. Generally, a shorter primer molecule requires a lower temperature to form a stable hybrid with a template. A primer sequence is not necessarily completely complementary with a template but must be complementary enough to hybridize with the template. Preferably, the 3′ end of the primer is aligned with a nucleotide (position 101) of each polymorphic site of SEQ ID NOS: 1-5. The primer is hybridized with a target DNA containing a polymorphic site and starts an allelic amplification in which the primer exhibits complete homology with the target DNA. The primer is used in pair with a second primer hybridizing with an opposite strand. Amplified products are obtained by amplification using the two primers, which means that there is a specific allelic form. The primer of the present invention includes a polynucleotide fragment used in a ligase chain reaction (LCR).

In the present invention, the allele-specific polynucleotide may be a probe. As used herein, the term ‘probe’ refers to a hybridization probe, that is, an oligonucleotide capable of sequence-specifically binding with a complementary strand of a nucleic acid. Such a probe may be a peptide nucleic acid as disclosed in Science 254, 1497-1500 (1991) by Nielsen et al. The probe according to the present invention is an allele-specific probe. In this regard, when there are polymorphic sites in nucleic acid fragments derived from two members of the same species, the probe is hybridized with DNA fragments derived from one member but is not hybridized with DNA fragments derived from the other member. In this case, hybridization conditions should be stringent enough to allow hybridization with only one allele by significant difference in hybridization strength between alleles. Preferably, the central portion of the probe, that is, position 7 for a 15 nucleotide probe, or position 8 or 9 for a 16 nucleotide probe, is aligned with each polymorphic site of the nucleotide sequences of SEQ ID NOS: 1-5. Therefore, a significant difference in hybridization between alleles may be caused. The probe of the present invention can be used in diagnostic methods for detecting alleles. The diagnostic methods include nucleic acid hybridization-based detection methods, e.g., southern blot. In a case where DNA chips are used for the nucleic acid hybridization-based detection methods, the probe may be provided as an immobilized form on a substrate of a DNA chip.

The present invention also provides a microarray for the detection of colorectal cancer, including the polynucleotide according to the present invention or the complementary polynucleotide thereof. The polynucleotide of the microarray may be DNA or RNA. The microarray is the same as a common microarray except that it includes the polynucleotide of the present invention.

The present invention also provides a diagnostic kit for the detection of colorectal cancer including the polynucleotide of the present invention. The diagnostic kit may include reagents necessary for polymerization, e.g., dNTPs, various polymerases, and a colorant, in addition to the polynucleotide according to the present invention.

The present invention also provides a method of diagnosing colorectal cancer in an individual, which includes: isolating a nucleic acid sample from the individual; and determining a nucleotide of at least one polymorphic site (position 101) within polynucleotides of SEQ ID NOS: 1-5 or complementary polynucleotides thereof. Here, when the nucleotide of the at least one polymorphic site of the sample nucleic acid is the same as at least one risk allele presented in Table 2, it is determined that the individual has a higher likelihood of being diagnosed as at risk of developing colorectal cancer.

The operation of isolating the nucleic acid sample from the individual may be carried out by a common DNA isolation method. For example, the nucleic acid sample can be obtained by amplifying a target nucleic acid by polymerase chain reaction (PCR) followed by purification. In addition to PCR, there may be used LCR (Wu and Wallace, Genomics 4, 560 (1989), Landegren et al., Science 241, 1077 (1988)), transcription amplification (Kwoh et al., Proc. Natl. Acad. Sci. USA 86, 1173 (1989)), self-sustained sequence replication (Guatelli et al., Proc. Natl. Acad. Sci. USA 87, 1874 (1990)), or nucleic acid sequence based amplification (NASBA). The last two methods are related with isothermal reaction based on isothermal transcription and produce 30 or 100-fold RNA single strands and DNA double strands as amplification products.

According to an embodiment of the present invention, the operation of determining the nucleotide of the at least one polymorphic site includes hybridizing the nucleic acid sample onto a microarray on which polynucleotides for diagnosis or treatment of colorectal cancer, including at least 10 contiguous nucleotides derived from the group consisting of nucleotide sequences of SEQ ID NOS: 1-5 and including a nucleotide of a polymorphic site (position 101), or complementary polynucleotides thereof are immobilized; and detecting the hybridization result.

A microarray and a method of manufacturing a microarray by immobilizing a probe polynucleotide on a substrate are well known in the pertinent art. Immobilization of a probe polynucleotide associated with colorectal cancer of the present invention on a substrate can be easily performed using a conventional technique. Hybridization of nucleic acids on a microarray and detection of the hybridization result are also well known in the pertinent art. For example, the detection of the hybridization result can be performed by labeling a nucleic acid sample with a labeling material generating a detectable signal, such as a fluorescent material (e.g., Cy3 and Cy5), hybridizing the labeled nucleic acid sample onto a microarray, and detecting a signal generated from the labeling material.

According to another embodiment of the present invention, as a result of the determination of a nucleotide sequence of a polymorphic site, when at least one nucleotide sequence selected from SEQ ID NOS: 1-5 containing respective risk alleles A, G, C, A, and A is detected, it is determined that the individual has a higher likelihood of being diagnosed as a colorectal cancer patient or as at risk of developing colorectal cancer. If more nucleotide sequences containing the risk alleles are detected in an individual, it may be determined that the individual has a much higher likelihood of being diagnosed as at risk of developing colorectal cancer.

### ADVANTAGEOUS EFFECTS

A protein of the present invention and a method of diagnosing colorectal cancer using the protein can be effectively used for diagnosis of colorectal cancer.

A polynucleotide of the present invention can be used for colorectal cancer-related applications such as diagnosis, treatment, or fingerprinting analysis of colorectal cancer.

A microarray and diagnostic kit including the polynucleotide of the present invention can be effectively used for the detection of colorectal cancer.

A method of analyzing polynucleotides associated with colorectal cancer of the present invention can effectively detect the presence or a risk of colorectal cancer.

### DESCRIPTION OF DRAWINGS

FIGS. 1 and 2 show that a protein identified by NCBI GenBank Accession No: XP_{—}033371 of the present invention is expressed at a remarkably high level in colorectal cancer cells, relative to other cancer cells and normal cells.

FIGS. 3 and 4 show that a protein identified by NCBI GenBank Accession No: XP_{—}033371 of the present invention is expressed in nucleoli during interphase and mitosis.

FIG. 5 shows the expression of a protein identified by NCBI GenBank Accession No: XP_{—}033371 of the present invention detected in nucleoli using an antibody against nucleolar protein B23.

FIG. 6 shows that a GFP-XP_{—}033371 fusion protein is associated with disassembly of nucleoli.

### BEST MODE

Hereinafter, the present invention will be described more specifically by Examples. However, the following Examples are provided only for illustrations and thus the present invention is not limited to or by them.

### EXAMPLES

### Example 1

### Analysis of Function of c14orf120 Gene

(1) Analysis of Expression Level of c14orf120 Gene in Cancer Cell Lines and Normal Cells

To evaluate the expression level of c14orf120 gene in various cancer cell lines and normal cells, cancer cell lines and normal cells were cultured and total RNAs were then isolated from the cultures. Then, RT-PCR was performed using oligonucleotide primers as set forth in SEQ ID NOS: 6 and 7 to amplify cDNA fragments of c14orf120 gene.

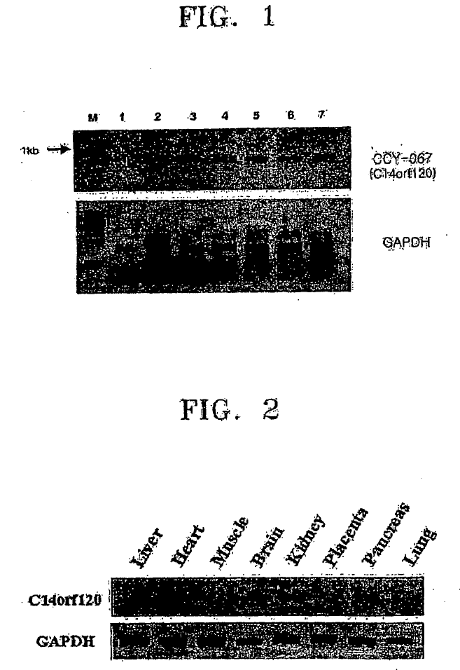

The results are shown in FIG. 1. As shown in FIG. 1, an expression level of c14 orf120 gene in colorectal cancer (HCT116, lane 5) was 2-8-fold higher than that in cervical adenocarcinoma (lane 2), osteosarcoma (lane 3), and liver cancer (lane 4, 6, and 7). In FIG. 1, lanes 1-7 are respectively IMR-90, Hela (human cervical adenocarcinoma cell lines), U2OS (osteosarcoma cell lines), Hep1 (human hepatoma cell lines), HCT116 (human colon carcinoma cell lines), Hep3B (human hepatoma cell lines), and Huh-7 (human hepatoma cells).

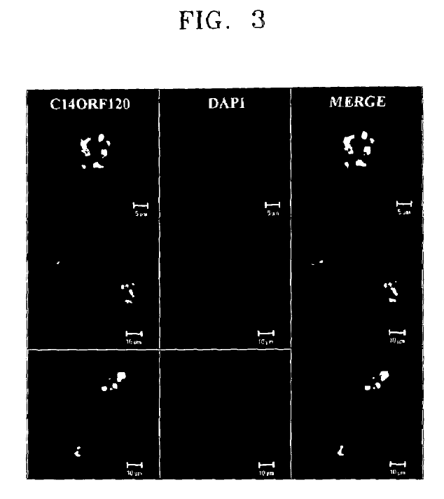

FIG. 2 shows an expression level of mRNAs of c14orf120 gene in various normal tissue cells by northern blotting using a multiple tissue northern blotting kit (BD Biosciences, USA). As shown in FIG. 2, the expression of c14orf120 gene in normal tissues was very weak.

(2) c14orf120 Gene Cloning and Construction of Expression Vectors for GFP Fusion Protein and Yeast 2-Hybrid Assay

cDNAs of c14orf120 gene were obtained by RT-PCR using, as a template, total RNAs isolated from SNU-449 cell lines, and c14orf120 gene-specific primers (SEQ ID NOS: 6 and 7), and sequence analysis was then performed. NCBI Blast searching based on the sequence analysis result revealed that the PCR products had the same sequence as c14orf120 gene.

Next, the PCR products were inserted into pGEM-T-Easy/c14orf120 vector (Promega, USA) by TA cloning. Then, the c14orf120 gene of the pGEM-T-Easy/c14orf120 vector was amplified by PCR, and the full-length c14orf120 DNAs were inserted into the EcoR I and BamH I restriction sites of a pEGFPC1 vector (BD Biosciences, USA) to thereby construct a pEGFPC1/c14orf120 which was an expression vector for GFP-c14orf120 fusion protein. On the other hand, the c14orf120 gene of the pGEM-T-Easy/c14orf120 vector was amplified by PCR, and the full-length c14orf120 DNAs were inserted into the EcoR I and BamH I restriction sites of a pGBKT7 vector (BD Biosciences, USA) to thereby obtain a pGBKT7/c14orf120 which was an expression vector for yeast 2-hybrid assay.

(3) Expression Position of c14orf120 Gene in Cells

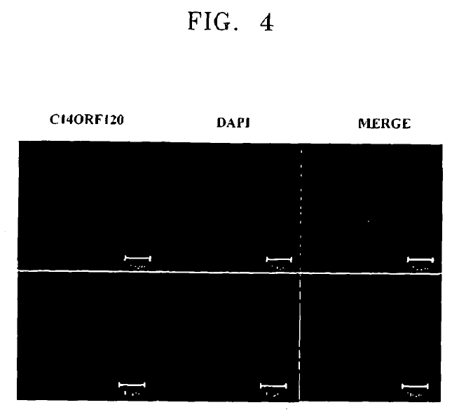

FIG. 3 shows fluorescence analysis results for transfected cells obtained by transfecting an expression vector for GFP-c14orf120 fusion protein, pEGFPC1/c14orf120, into U2OS cell lines during interphase. As shown in FIG. 3, the GFP-c14orf120 fusion protein was expressed in nucleoli. FIG. 4 shows fluorescence analysis results for transfected cells obtained by transfecting an expression vector for GFP-c14orf120 fusion protein, pEGFPC1/c14orf120, into U2OS cell lines during mitosis. As shown in FIG. 4, the c14orf120-GFP fusion protein was positioned in chromosome. In FIGS. 3 and 4, DAPI (4′,6-diamidino-2-phenylindole) is a staining reagent for visualization of chromosome and MERGE is a merge image of GFP-c14orf120 and DAPI used for accurately detecting the expression position for GFP-c14orf120 fusion protein in cells.

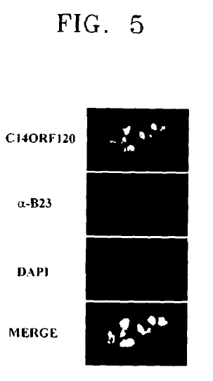

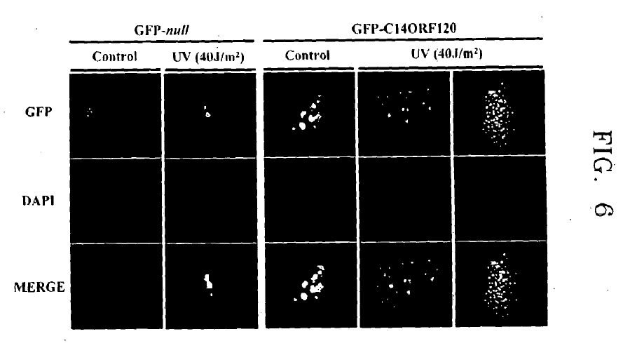

FIG. 5 shows the position of c14orf120 gene in cells, detected using an antibody against nucleolar protein B23. That is, the positions of B23, known as a nucleolar protein, and c14orf120 in cells were observed by an immunofluorescence assay using a B23 antibody. For this, cultured U2OS cell lines were transfected with the pEGFPC1/c14orf120 vector, fixed, and incubated with the B23 antibody at room temperature for one hour. After cell washing, the transfected cell lines were again incubated with a secondary antibody for 40 minutes and treated with DAPI. The cell lines were observed by fluorescence microscopy or confocal laser scanning microscopy. The observation results revealed that B23 and c14orf120 were distributed in the same nucleolar sites.

FIG. 6 shows that the GFP-XP_{—}033371 fusion protein is associated with disassembly of nucleoli. That is, FIG. 6 shows fluorescence microscopic images for exposure of pEGFPC1/c14orf120-transiently transfected U2OS cells to UV (40 J/m²) for 6 hours. For this, the U2OS transfected cells expressing GFP-c14orf120 were exposed to UV (40 J/m²) and fixed, and a change in the cells was observed. In FIG. 6, GFP-null is a transfected cell line expressing only GFP, and GFP-C14ORF120 is a transfected cell line expressing the GFP-c14orf120 fusion protein. With respect to the GFP-c14orf120 cell line, foci were wholly formed over a cell nucleus due to cell damage by UV, unlike the GFP-null cell line. As shown in FIG. 6, the disassembly of nucleoli was observed in the pEGFPC1/c14orf120-transfected cell line.

(4) Detection of Protein Interacting with c14orf120 Gene

Detection of proteins interacting with c14orf120 was done using the expression vector for yeast 2-hybrid assay, pGBKT7/c14orf120. The experiments were performed according to the manufacturer's instruction using a commercially available kit (BD Matchmarker™ Systems). The pGBKT7-c14orf120 vector was inserted into yeast AH109 cells to construct transfectants. The transfectants were hybridized with yeast Y187 cells in which a human testis cDNA library vector was inserted. After 24 hours of the hybridization, the diploid yeast cells were washed and uniformly plated onto an amino acid (Trp, Leu, His, Ade) restriction medium-containing plate. After about 5-7 days, cell colonies were harvested, and yeast cells containing genes interacting with c14orf20 were selected from the colonies by beta-galactosidase assay. The nucleotide sequences of the yeast cell genes were analyzed by colony PCR.

The results are presented in Table 1. As shown in Table 1, total 18 positive colonies were found, i.e., C1QBP1, YB-1, ten AATFs, and six Myc-binding protein-associated proteins.

The above results reveal that the protein of NCBI GenBank Accession No: XP_{—}033371 is present in nucleoli and has a nucleolus-associated function. Judging from the fact that the protein is present in chromosome during mitosis, the protein has a function related to cell cycle. In addition, the protein of NCBI GenBank Accession No: XP_{—}033371 and AATF are functionally associated with each other. It is known that AATF is a tumor protein binding with RB and inhibiting the growth inhibitory effect of RB. Thus, the protein of NCBI GenBank Accession No: XP_{—}033371 binds with AATF to facilitate the binding of AATF with RB or cooperates with AATF to thereby induce tumorigenesis.

In addition to these results, the present inventors investigated the association of SNPs in c14orf120 gene region with colorectal cancer as follows.

### Example 2

### Analysis of Occurrence Frequency of SNPs of c14orf120 Gene

In this Example, DNA samples were extracted from blood streams of a patient group consisting of 300 Korean persons that had been diagnosed as colorectal cancer patients and had been being under treatment and a normal group consisting of 300 Korean persons which were of the same age as those in the patient group and had no colorectal cancer symptoms, and occurrence frequencies of SNPs in c14orf120 gene were evaluated. SNPs used in this Example were rs7151139, rs10142383, rs2236261, rs6573195 and rs2295706 selected from a known database (NCBI dbSNP:http://www.ncbi.nlm.nih.gov/SNP/). Primers hybridizing with sequences around the selected SNPs were used to assay nucleotides of SNPs in the DNA samples.

1. Preparation of DNA Samples

DNA samples were extracted from blood streams of colorectal cancer patients and normal persons. DNA extraction was performed according to a known extraction method (Molecular cloning: A Laboratory Manual, p 392, Sambrook, Fritsch and Maniatis, 2nd edition, Cold Spring Harbor Press, 1989) and the specification of a commercial kit manufactured by Centra system. Among extracted DNA samples, only DNA samples having a purity (measured by A₂₆₀/A₂₈₀ nm ratio) of at least 1.7 were used.

2. Amplification of Target DNAs

Target DNAs, which were predetermined DNA regions containing SNPs to be analyzed, were amplified by PCR. The PCR was performed by a common method as the following conditions. First, target genomic DNAs were diluted to concentration 2.5 ng/ml. Then, the following PCR mixture was prepared.

Water (HPLC grade) 2.24□

10× buffer (15 mM MgCl₂, 25 mM MgCl₂) 0.5□

dNTP Mix (GIBCO) (25 mM for each) 0.04□

Taq pol (HotStar) (5 U/□) 0.02□

Forward/reverse primer Mix (1 μM for each) 0.02[

DNA 1.00└

Total volume 5.00□

Here, the forward and reverse primers were designed based on upstream and downstream sequences of SNPs in known database. These primers are listed in Table 4 below.

The condition of PCR were as follows: incubation at 95° C. for 15 minutes, at 95° C. for 30 seconds, at 56° C. for 30 seconds, and at 72° C. for 1 minute, repeated 45 times; and finally incubation at 72° C. for 3 minutes and storage at 4° C.

3. Analysis of SNPs in Amplified Target DNA Fragments

Analysis of SNPs in the amplified target DNA fragments was performed using a homogeneous MassEXTEND (hME) technique available from Sequenom. The principle of the MassEXTEND technique is as follows. First, primers (also called as ‘extension primers’) ending immediately one base before SNPs within the target DNA fragments were designed. Then, the primers were hybridized with the target DNA fragments and DNA polymerization was initiated. At this time, a polymerization solution contained a reagent (e.g., ddTTP) terminating the polymerization immediately after the incorporation of a nucleotide complementary to a first allelic nucleotide (e.g., A allele). In this regard, when the first allele (e.g., A allele) exists in the target DNA fragments, products in which only a nucleotide (e.g., T nucleotide) complementary to the first allele is extended from the primers will be obtained. On the other hand, when a second allele (e.g., G allele) exists in the target DNA fragments, a nucleotide (e.g., C nucleotide) complementary to the second allele is added to the 3′-ends of the primers and then the primers are extended until a nucleotide complementary to the closest first allele nucleotide (e.g., A nucleotide) is added. The lengths of products extended from the primers were determined by mass spectrometry. In this way, alleles present in the target DNA fragments could be identified. Illustrative experimental conditions were as follows.

First, unreacted dNTPs were removed from the PCR products. For this, 1.53□ of distilled water, 0.17[ of HME buffer, and 0.30□ of shrimp alkaline phosphatase (SAP) were added and mixed in 1.5 ml tubes to prepare SAP enzyme solutions. The tubes were centrifuged at 5,000 rpm for 10 seconds. Thereafter, the PCR products were added to the SAP solution tubes, sealed, incubated at 37° C. for 20 minutes and then 85° C. for 5 minutes, and stored at 4° C.

Next, homogeneous extension was performed using the target DNA fragments as templates. The compositions of reaction solutions for the extension were as follows.

Water (nanoscale distilled water) 1.728□

hME extension mix (10× buffer containing 2.25 mM d/ddNTPs) 0.200□

Extension primers (100 μM for each) 0.054□

Thermosequenase (32 U/␣) 0.018␣

Total volume 2.00□

The reaction solutions were thoroughly stirred and subjected to spin-down centrifugation. Tubes or plates containing the resultant solutions were compactly sealed and incubated at 94° C. for 2 minutes, followed by 40 thermal cycles at 94° C. for 5 seconds, at 52° C. for 5 seconds, and at 72° C. for 5 seconds, and storage at 4° C. The homogeneous extension products thus obtained were washed with a resin (SpectroCLEAN™). Nucleotides of polymorphic sites in the extension products were assayed using mass spectrometry, MALDI-TOF (Matrix Assisted Laser Desorption and Ionization-Time of Flight). The MALDI-TOF is operated according to the following principle. When an analyte is exposed to a laser beam, it flies toward a detector positioned at the opposite side in a vacuum state, together with an ionized matrix. At this time, the time taken for the analyte to reach the detector is calculated. A material with a smaller mass reaches the detector more rapidly. The nucleotides of SNPs in the target DNA fragments were determined based on a difference in mass between the DNA fragments and known SNP sequences. Primers used in the amplification and extension or the target DNAs are listed in Table 4 below.

TABLE 4

| | Amplification primer (SEQ ID NO.) | | Extension primer |
|---|---|---|---|
| Marker | Forward primer | Reverse primer | (SEQ ID NO.) |
| CCK061 | 8 | 9 | 10 |
| CCK162 | 11 | 12 | 13 |
| CCY_067 | 14 | 15 | 16 |
| CCY_202 | 17 | 18 | 19 |
| CCY_205 | 20 | 21 | 22 |

The results for the determination of polymorphic sequences of the target DNAs using the MALDI-TOF are shown in Table 2 above. Each allele may exist in the form of homozygote or heterozygote in an individual. However, in population, the relative frequency of homozygote and heterozygote is statistically insignificant. According to Mendel's Law of inheritance and Hardy-Weinberg Law, a genetic makeup of alleles constituting a population is maintained at a constant frequency. When the genetic makeup is statistically significant, it can be considered to be biologically meaningful.

### INDUSTRIAL APPLICABILITY

A protein of the present invention and a method of diagnosing colorectal cancer using the protein can be effectively used for diagnosis of colorectal cancer.

A polynucleotide of the present invention can be used for colorectal cancer-related applications such as diagnosis, treatment, or fingerprinting analysis of colorectal cancer.

A microarray and diagnostic kit including the polynucleotide of the present invention can be effectively used for the detection of colorectal cancer.

A method of analyzing polynucleotides associated with colorectal cancer of the present invention can effectively detect the presence or a risk of colorectal cancer.

1. An isolated nucleolar protein having an amino acid sequence of NCBI GenBank Accession No. XP_{—}033371.

2. A method of diagnosing colorectal cancer in an individual, which comprises measuring an expression level of a protein having an amino acid sequence of NCBI GenBank Accession No. XP_{—}033371 in the individual.

3. The method of claim 2, wherein the expression level of the protein is determined by measuring the amount of the protein in cells derived from the individual or the amount of mRNA encoding the protein.

4. The method of claim 2, wherein when the expression amount of the protein is 20% or more higher than that in normal cells, it is determined that the individual has a higher likelihood of being diagnosed as a colorectal cancer patient or as at risk of developing colorectal cancer.

5. A polynucleotide comprising at least 10 contiguous nucleotides of a nucleotide sequence selected from the group consisting of nucleotide sequences of SEQ ID NOS: 1-5 and comprising a nucleotide at position 101 of the nucleotide sequence, or a complementary polynucleotide thereof.

6. A polynucleotide which is hybridized with the polynucleotide of claim 5 or the complementary polynucleotide thereof.

7. The polynucleotide of claim 5, which is 10 to 100 nucleotides in length, or the complementary polynucleotide thereof.

8. The polynucleotide of claim 5, which is a primer or a probe.

9. A microarray comprising the polynucleotide of claim 5 or the complementary polynucleotide thereof.

10. A diagnostic kit for the detection of colorectal cancer, which comprises the polynucleotide of claim 5 or the complementary polynucleotide thereof.

11. A method of diagnosing colorectal cancer in an individual, which comprises:
isolating a nucleic acid sample from the individual; and
determining a nucleotide of at least one polymorphic site (position 101) within polynucleotides of SEQ ID NOS: 1-5 or complementary polynucleotides thereof.

12. The method of claim 11, wherein the operation of determining the nucleotide of the at least one polymorphic site comprises:
hybridizing the nucleic acid sample onto a microarray on which the polynucleotide of claim 5 or its complementary polynucleotide is immobilized; and
detecting a hybridization result.

13. The method of claim 11, wherein when at least one nucleotide sequence selected from SEQ ID NOS: 1-5 containing respective polymorphic nucleotides A, G, C, A, and A is detected, it is determined that the individual has a higher likelihood of being diagnosed as a colorectal cancer patient or as at risk of developing colorectal cancer.

14. The polynucleotide of claim 6, which is 10 to 100 nucleotides in length, or the complement thereof.
